(19) United States
(12) Patent Application Publication
(10) Pub. No.: US 2022/0039568 A1
(43) Pub. Date: Feb. 10, 2022

(54) STRESS RELIEVING INSERT FOR BEDSHEETS

(71) Applicant: Peter Cordani, Jupiter, FL (US)

(72) Inventor: Peter Cordani, Jupiter, FL (US)

(21) Appl. No.: 17/395,589

(22) Filed: Aug. 6, 2021

Related U.S. Application Data
(60) Provisional application No. 63/061,841, filed on Aug. 6, 2020.

(51) Int. Cl. A47G 9/02

(52) U.S. Cl. CPC A47G 9/0238; A47G 2009/008

(57) ABSTRACT

A bedsheet having a stress relieving foam insert around a sewn edge. The foam insert is preferably constructed from closed-cell polyurethane foam rubber and may include a fluid bladder when the foam insert is formed for segments. The fluid bladder for use in enlarging the foam inserts, and may include reed apertures positioned between segments wherein audible sounds are created when fluid is transferred between segments. The squeezing of the foam inserts providing stress relief to an individual and promoting restful sleep. The foam inserts attached to bedsheet to prevent misplacement.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 63/061,841 entitled “STRESS RELIEVING INSERT FOR BEDSHEETS AND BLANKETS” filed on Aug. 6, 2020 the contents of which is incorporated herein by reference in its entirety.

### FIELD OF THE INVENTION

The present invention relates to bedsheets, and in particular, to a stress relieving foam insert formed in a hem of a bedsheet for use in achieving stress relief, thereby promoting restful sleep by squeezing and releasing the foam insert.

### BACKGROUND OF THE INVENTION

Many people suffer with the inability to relax and fall asleep for many reasons, including the stresses of everyday life, intrusion of thoughts, and muscle and joint aches and pains. One method of relaxing is the use of a “stress ball” which consists of a semi-soft foam ball that an individual can squeeze by hand repeatedly. The stress ball is proven to provide therapeutic relief from mental or physical stress by repetition of squeezing and releasing the foam ball.

While the stress ball is most beneficial, the use of such a stress ball while lying in bed can be impractical. Unless the stress ball is held in a stationary position, it can be easily displaced. For instance, if the stress ball is used to help an individual fall asleep, when the individual does fall asleep the ball will be released from the individual's hand when sleep is bestowed upon the individual. If the individual is restless, the stress ball can be displaced by the time the individual awakens. More realistically, the individual who has a restless night may want to access the stress ball during the night. In this example, the individual may need to search for the stress ball in the darkness, wherein the ball could be entangled within the bedsheets. More critically the stress ball could fall off the bed and, should the individual need to leave the bed, the individual runs the risk on stepping upon the stress ball which could easily lead to a fall and injury. Most individuals do awaken before morning light to use a bathroom, check on strange noises, check on children, obtain water, snack and so forth. For this reason it is not recommended that a stress ball be used while the individual is in a bed. Tethering a stress ball to a bed limits movement of the ball and creates even further safety complications.

### PRIOR ART REFERENCES

U.S. Pat. No. 5,708,999 discloses an adjustable support pillow having at least one bolster portion along one edge of the support pillow, and an inflatable under-pillow. The under-pillow comprises at least one row of a plurality of parallel inflatable air chambers. Each air chamber within a row is in fluid communication with each adjacent air chamber in the same row. Each of the rows is in fluid communication with a header and the header is connectable to an air source by a nozzle.

U.S. Pat. No. 7,513,001 discloses a pillow with an attached blanket. The pillow comprises a body with one or more inwardly-turned arms. One edge of the blanket is attached to the pillow, and a pouch is formed in the pillow so that the blanket is stowable inside. A pocket may be included for holding an MP3 player or other accessory. A flap wraps around the body to cover the pouch opening and the accessory pocket when the blanket is stowed.

U.S. Pat. No. 8,234,732 discloses an orthopedic pillow having a plurality of foam layers. The foam layers include a top foam layer made of memory foam constructed and arranged to receive a user's head. A middle foam layer is made of latex foam, the middle layer defining a central opening, and a bottom layer made of latex foam. The orthopedic pillow also has a high density foam tube affixed to an edge of the top foam layer. The high density foam tube is constructed and arranged to support the user's neck.

U.S. 2019/0320827 discloses a sensory stress relief aid having a pillow with a plurality of different surfaces disposed on an outer layer. Each surface corresponds to a different tactile feel, such as velvet or fur. Additionally, the outer layer encloses a plurality of inner layers disposed within the pillow, wherein each inner layer comprises a different degree of firmness. A motor attached to an inflatable bladder disposed within the plurality of inner layers is configured to alternately inflate and deflate to simulate breathing. At least one speaker is included and adapted to emit soothing sounds such as nature noises and guided meditation instructions.

U.S. Pat. No. 10,314,418 discloses a pillow that includes a first pillow half having an outer surface and an inner surface having a first cavity formed therein filled with a first filler material, and a second pillow half having an outer surface and an inner surface having a second cavity formed therein filled with a second filler material.

U.S. Pat. No. 10,292,509 discloses a pillow for seating on a base surface. The pillow includes a top surface having a negative parabolic shape or a convex shape relative to the base surface and a bottom surface having a convex shape relative to the base surface. The pillow includes up to five zones I-V, with a center zone I being designed for side sleepers, the two adjacent zones II, III being designed for back sleepers, and the two adjacent, outer zones IV, V being designed for stomach sleepers. The pillow may have different sections and parts in order to vary the feel of the pillow.

What is needed in the industry is a bedsheet having stress ball(s) formed integral thereto, namely a foam insert providing the same therapeutic relief to any individual by placing the insert in one of the hems of the bedsheet. The insert constructed and arranged to be used for squeezing and releasing in a stationary location to help an individual relieve stress while in bed. If the individual wakes up through the night, the stress relieving foam insert is readily available by simply grasping the insert positioned within the hem of the bedsheet.

### SUMMARY OF THE INVENTION

Disclosed is a bedsheet having a foam insert secured to the bedsheet by a hem, the foam insert is available for use in providing a method for stress relief.

An objective of the invention is to provide a stress relieving aid for use in a bed wherein the repeated squeezing of the stress relieving aid enables an individual to concentrate on the repeated motion which is recognized to alleviate stress and promote restful sleep.

Another objective of the instant invention is to provide a stress relieving foam insert that is readily accessible while in a bed, the foam insert being attached within a hem of a bedsheet so as to prevent misplacement of the foam insert.

Another objective of the instant invention is teach the placement of a stress relieving foam insert device so as to prevent the device from leaving the confines of a bed, such as falling on the floor where an individual could trip on the device during the night.

Still another objective of the invention is to teach the use of a stress relieving foam insert for use in a bedsheet, blanket, pillow cover, comforter, covers, covering, and the like bedding materials.

Other objectives and advantages of this invention will become apparent from the following description, taken in conjunction with any accompanying drawings wherein are set forth, by way of illustration and example, certain embodiments of this invention. Any drawings contained herein constitute a part of this specification, include exemplary embodiments of the present invention, and illustrate various objectives and features thereof.

### BRIEF DESCRIPTION OF THE DRAWINGS

FIG. 1 is a perspective view of a bedsheet with a foam insert placed within a hem;

FIG. 2 is a perspective view of a foam insert; and

FIG. 3 is a perspective view of a bedsheet with a single foam insert secured to one end and a plurality of foam inserts secured to an opposite end.

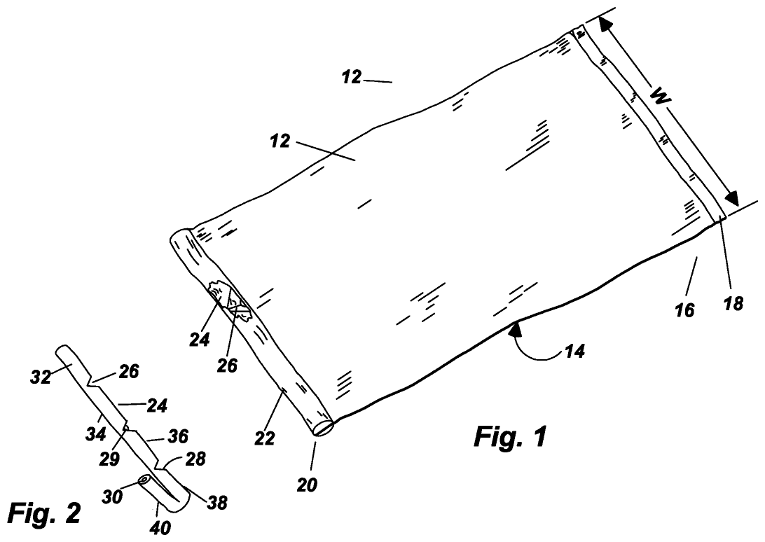

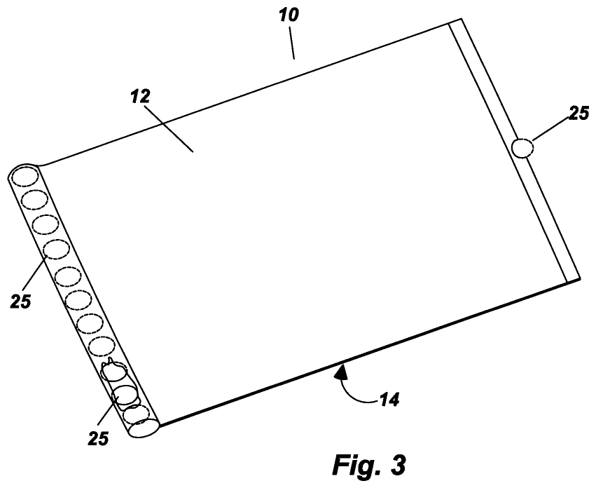

### DESCRIPTION OF THE PREFERRED EMBODIMENT

Detailed embodiments of the instant invention are disclosed herein; however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

Referring to the Figures, disclosed is the bedsheet 10 of the instant invention defined by a rectangular piece of flat-woven textile having an upper surface 12 and a lower surface 14, bounded along a first end 16 by hem 18, and a second end 20 by hem 22. The hem is defined as the sewn edge formed on each end of the bedsheet which has been turned under and sewn together. The term bedsheet may be interchanged with the term “blanket” or “pillow cover” for purposes of this specification. It will be understood by one skilled in the art that the invention can be placed in the sewn hem of a pillow case, blanket, comforter, covers, covering, and the like bedding materials.

The hem 22 receives a stress foam insert 24 which, in one embodiment, is about two inches in diameter and extends along a width W of the bedsheet. The foam insert being captured in the portion of the bedsheet that has been turned under and sewn together. The stress foam insert 24 is preferably constructed of segments which allow folding of the bedsheet when not in use, and facilitate washing of the bedsheet. Alternatively the foam insert can be temporarily removed from the hem area by use of a zipper, hook and loop, or snap fastener allowing removal of the foam insert for washing or storage. Segments are formed by a plurality of valleys 26 that allow folding of the foam insert 24. The valleys 26 are constructed and arranged to nest providing a compact shape for washing or storage. Each valley 26 has an offset sidewall 28, allowing the foam insert to fold in a predetermined pattern. In another embodiment the stress form inserts 25 are of a circular shaped, formed from multiple ball shaped foam inserts, or a continuous form insert with segments resembling a ball shape.

In another embodiment, a bladder 30 can be positioned within the stress foam insert 24 which, upon inflation, will provide the foam insert 24 with additional resistance when squeezed. The inflated bladder can be placed in individual sections 32, 34, 36, 38 and 40 wherein fluid, either air or liquid, operates to fill an inner section of the foam insert 24 providing both enlargement and resistance functions. The fluid providing a variable resistance and, depending upon the interconnecting reed aperture 29 design, provide a smoothing sound caused by the passage of air or liquid between sections. For instance, the squeezing of one section can cause the displacement of air that, with the appropriate reed placed between segments, will make the sound of whales at sea. In an embodiment using liquid, passage of liquid can be made to sound like ocean waves. The liquid can be water or preferably a gel formed from a super absorbent polymeric and water composition. The composition utilizes a biodegradable super absorbent aqueous based polymer, namely a cross-linked modified polyacrylamides/potassium acrylate or polyacrylamides/sodium acrylate. Other suitable polymers include, albeit are not limited to, carboxy-methylcellulose, alginic acid, cross-linked starches, and cross-linked polyaminoacids.

The bedsheet can be made of linen, cotton, synthetics, silk or the like. The foam insert is made from a biocompatible, non-toxic material capable of providing a desired resistance. In the preferred embodiment, the foam is selected from the group consisting of closed-cell polyurethane foam, food-grade rubber, natural rubber, silicone rubber, silicone and latex, or any other FDA-approved rubber, latex or suitable polymer.

The resistance provided is similar to a stress ball, and can be measured as the force per unit area required to deform the device so that the outer circumference of the foam insert can be squeezed to a point where the opposing side walls just about touch each other. The foam is soft enough that laying on it would not disturb sleep, but would provide progressively sufficient resistance, wherein an elderly individual would be able to cause partial collapse of the foam, and a muscular individual would be challenged to completely collapse the foam. The resistance can be measured by placing the foam on a scale and pressing down on the foam with a one inch square surface area to determine the weight on the scale at which the top and bottom walls of the foam nearly touch each other. When the scale is calibrated in pounds, the resistance associated with the foam is expressed as pounds per square inch (psi). When resistance is measured in this manner, the preferred foam will have a resistance between about 20 psi and about 100 psi. Preferred resistance is between about 40 psi, but can vary based upon the diameter of the foam, if round, or other shape employed. The resistance of a device depends on the size of the foam, and the material from which the foam is made. One of skill in the art can readily vary these parameters to produce the devices of the invention.

In use, an individual would install the bedsheet having the stress foam insert in the ordinary and conventional manner by placement over a bed. If the stress foam insert is positioned within a blanket, the blanket is typically placed over the bedsheet. With the stress relieving foam formed in the end of the bedsheet, an individual can grasp or clench the foam with multiple repetitions; such repetitions can be with a planned number of sets or until the individual falls asleep. Should the individual wake up during the night and become restless, the individual will easily find the stress relieving foam, as it will remain in the hem of the bedsheet or blanket.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs.

It is to be understood that while a certain form of the invention is illustrated, it is not to be limited to the specific form or arrangement herein described and shown. It will be apparent to those skilled in the art that various changes may be made without departing from the scope of the invention, and that the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A stress relief bedsheet comprising: a bedsheet formed from a substantially rectangular piece of fabric material having a proximal end terminating with a first hem, and a distal end terminating with a second hem; and at least one foam insert positioned within one said hem.

2. The stress relief bedsheet according to claim 1 wherein said foam insert comprised of a plurality of segments, said segments including valleys constructed and arranged to allow a pattern folding of said form insert.

3. The stress relief bedsheet according to claim 2 including at least one bladder formed in at least one segment, said bladder for receipt of a fluid.

4. The stress relief bedsheet according to claim 3 including a reed aperture positioned between a first bladder segment and a second bladder segment, said reed aperture capable of producing an audible sound when fluid is transferred between said first bladder segment and second bladder segment.

5. The stress relief bedsheet according to claim 4 wherein said fluid is a liquid.

6. The stress relief bedsheet according to claim 5 wherein said liquid is a gel.

7. The stress relief bedsheet according to claim 6 wherein said gel is formed from a super absorbent polymeric and water composition.

8. The stress relief bedsheet according to claim 7 wherein said super absorbent polymeric is a cross-linked modified polyacrylamides/potassium acrylate or polyacrylamides/sodium acrylate.

9. The stress relief bedsheet according to claim 1 wherein said stress foam insert is about two inches in diameter.

10. The stress relief bedsheet according to claim 1 wherein said foam insert is selected from the group consisting of: closed-cell polyurethane foam rubber, food-grade rubber, natural rubber, silicone rubber, silicone and latex, or a polymer.
